SEQUENCE LISTING (2 sequences)

SEQ ID NO: 1 - protein, 6 aa, Artificial Sequence
  synthetic peptide
AVCGTG

SEQ ID NO: 2 - protein, 6 aa, Artificial Sequence
  synthetic peptide
ACTGVG

(19) United States
(12) Patent Application Publication
(10) Pub. No.: US 2016/0256535 A1
(43) Pub. Date: Sep. 8, 2016

(54) HEXAPEPTIDE AND USES THEREOF

(71) Applicant: Aldo MANCINI, Napoli (NA) (IT)

(72) Inventor: Aldo MANCINI, Napoli (NA) (IT)

(21) Appl. No.: 15/026,819

(22) PCT Filed: Oct. 2, 2014
(86) PCT No.: PCT/EP2014/071202

§ 371 Date: Apr. 1, 2016

(30) Foreign Application Priority Data
Oct. 3, 2013 (IT) RM2013A000538

(51) Int. Cl. A61K 39/00; A61K 33/24; C12N 9/02; A61K 47/48

(52) U.S. Cl. CPC A61K 39/0011; A61K 47/4833; A61K 33/24; C12N 9/0089; A61K 2039/575; C12Y 115/01001

(57) ABSTRACT

The present invention concerns a hexapeptide and its medical and/or diagnosis uses, in particular for the diagnosis and/or treatment of cancer.

### FIELD OF THE INVENTION

The present invention concerns a hexapeptide and its medical and/or diagnosis uses, in particular for the diagnosis and/or treatment of cancer.

### BACKGROUND ART

The significance of chromosomal abnormalities in tumor development has been demonstrated since the beginning of the last century. However, only in the last 20 years the development of cytogenetics and molecular biology have proved that the primary cause of tumors lies in chromosomal instability which leads to alterations of oncogenes and tumor suppressor genes and changes of signal transduction, or alteration of the cell cycle, Apoptosis, Telomerase, of alterations that affect the mechanisms of DNA repair, and so forth (1-5).

In a recent study on the characterization of a cell line of liposarcoma stabilized in continuous culture (LSA), it was discovered that these cells release in their culture medium several proteins including an isoform of Manganese Superoxide Dismutase (LSA-type-MnSOD). It was proved that this protein, although having its specific enzymatic activity, consisting in the transformation of free radicals into hydrogen peroxide, common to all the other SODs, features structural and functional characteristics that differentiate it from the MnSOD expressed in myeloid leukemia cells U937 (6).

In this respect LSA-Type-MnSOD has, for example, a molecular weight of about 30 kDa, whereas the wild-type protein has a molecular weight of 26 KDa. The LSA-type-MnSOD is released from the cells in the culture medium with the whole leader peptide firmly bound to the N-terminal portion of the molecule. Nevertheless, the molecule is enzymatically active, whereas the native one, in order to become mature, would have to lose the leader peptide. This always occurs before entering the inner matrix of the mitochondrion.

Furthermore, if LSA-type-MnSOD is injected in vivo or added to cultured cells in vitro, due to the presence of the leader peptide which acts as a molecular carrier, it is able to penetrate all the cells exerting its enzymatic activity of transformation of the free radicals to hydrogen peroxide, which the catalase will convert into molecular oxygen and water.

The antitumor action of LSA-typeMnSOD is linked to the fact that cancer cells produce a lower quantity of catalase (from 10 to 50 times) compared to the one produced by normal cells. Therefore, they will not be able to convert the peroxide hydrogen formed as a result of the LSA-type-MnSOD. This will produce a high concentration of peroxide that will exceed the threshold of toxicity only in tumor cells that die by apoptosis (7).

This aspect confirms that LSA-type-MnSOD is cytotoxic in a specific and selective way for tumor cells only.

A recombinant form of LSA-type-MnSOD, the (rMnSOD), obtained from a cDNA derived from LSA cells, has the same functional characteristics of the extractive protein and, as the extractive molecule, still has by the N-terminal portion of the molecule the leader peptide composed of 24 amino acids. This protein is enzymatically active as well(8).

The inventor of the present invention has previously shown that the leader peptide of 24 amino acids (aa.), present in the recombinant molecule rMnSOD, was the carrier element able to penetrate cells followed by the entire protein sequence (7).

The documents WO 2010/004513, Borrelli et al. 2010 (14), Borrelli et al. 2012 (15) and WO 2006/117250 disclose the leader peptide of MnSOD or a derivative thereof in its free or conjugated form.

### SUMMARY OF THE INVENTION

In the present invention the inventor has investigated the function of this leader peptide, mainly identifying the amino acids of the leader sequence responsible for the phenomenon of internalization. From this research, it was found that only six of the 24 aa. (AVCGTG, SEQ ID NO. 1) that compose the leader sequence, are responsible for the internalization of the peptide in the cells. Such a result may be achieved by binding to a receptor of the cell membrane, yet unidentified.

When penetrating into the cells, the hexapeptide transports the entire protein and, may also carry any molecule conjugated to it.

The hexapeptide of the invention can be used as a molecular carrier for therapeutic purposes, for example by binding to molecules with therapeutic activity and sending the construct into damaged cells. The hexapeptide of the invention can also be used as a tumor marker for diagnostic purposes, by binding to a γ or β-emitting radioactive isotope capable of revealing or to kill a tumor. Further, the hexapeptide of the invention can be used for immune purposes to elicit an immunological response.

For instance, if the peptide binds an antigen that causes a disease, such a construct could be sent in leukocyte cells, withdrawn from the ill patient and later be cultured. Once the construct will have entered the patient's white blood cells in vitro, they would be then re-injected in the donor, this time enriched with antigen. The final result of this strategy is that the white blood cells that are carriers of the construct, and of the antigen, will present to the Helper T-lymphocytes the antigen, triggering the production of monoclonal antibodies against that antigen, directly in the patient.

The present invention provides a peptide consisting of the sequence AVCGTG (SEQ ID No. 1). In one embodiment the peptide is conjugated, bound or linked to a molecule.

Preferably the molecule is selected from the group consisting of: a therapeutic agent, a dye, a chelating agent, an antigen, a peptide, a metal, a radioactive isotope λ and β emitting, or an antibody molecule.

In one embodiment the therapeutic agent is selected from the group consisting of: chemotherapeutic agent, antibiotic agent, anti-inflammatory agent, anthistamine agent, antiparasitic agent, antiviral agent, antitumor antibody.

The chemotherapeutic agent may be any chemotherapeutic agent and may be selected by the skilled person in the art according to common knowledge. Preferably the chemotherapeutic agent is selected from the group consisting of: cisplatin, idarubicin, epirubicin, cytarabine, farmarubicin, etoposside.

The antibiotic agent may be any antibiotic agent and may be selected by the skilled person in the art according to common knowledge. Preferably the the antibiotic agent is selected from the group consisting of: b-lactam, quinolone antibiotics.

In one embodiment, the peptide of the invention alone or conjugated is for medical and/or diagnostic use.

Preferably the peptide of the invention alone or conjugated is for use in the treatment and/or prevention and/or diagnosis of a tumor pathology.

Preferably the tumor pathology is a leukemia.

The present invention also provides the use of the peptide of the invention as a carrier for a molecule to be vehiculated to a target site.

The target site may be any site where a molecule needs to be vehiculated. Preferably the target site is selected from the group consisting of: tumor cell, leukocyte, gamete, normal cell, stem cell, bacteria, preferably the bacteria is resistant to at least one therapeutic agent. A normal cell is for instance a cell of a healthy subject. The target site may also be an entire organ. The stem cell may be an adult or embryo stem cell. Such cell is obtained without the destruction of an embryo.

In one embodiment, the peptide of the invention alone or conjugated is for use to induce an immunological response.

The present invention also provides the use of the peptide of the invention alone or conjugated for the production of antibodies.

The present invention provides a molecule selected from the group consisting of:
a nucleotide sequence coding for the peptide consisting of the sequence AVCGTG (SEQ ID No. 1) or ortolog nucleotide sequence thereof;
a vector comprising said nucleotide sequence;
a host cell genetically engineered expressing said peptide.

The present invention provides a composition comprising a molecule and acceptable excipients wherein the molecule is selected from the group consisting of:
a) peptide consisting of the sequence AVCGTG (SEQ ID No. 1);
b) a nucleotide sequence coding for the peptide consisting of the sequence AVCGTG (SEQ ID No. 1) or ortholog nucleotide sequence thereof;
c) a vector comprising said nucleotide sequence;
d) a host cell genetically engineered expressing said peptide.

Preferably the peptide is conjugated to a molecule.

The peptide of the invention alone or conjugated to a molecule may also be encapsulated in nanoparticles, in liposomes or other means.

The present invention provides the above composition for medical and/or diagnostic use. Preferably for use in the treatment and/or prevention and/or diagnosis of a tumor. Preferably the tumour is a leukemia.

The present invention provides the use of the above composition as a carrier for a molecule to be vehiculated to a target site. Preferably the target site is selected from the group consisting of: tumor cell, leukocyte, gamete, normal cell, stem cell, bacteria, preferably the bacteria is resistant to at least one therapeutic agent. By resistance it is meant a decreased or an absent response to the therapeutic agent, in particular an antibiotic.

The present invention provides the use of the above composition wherein the peptide is alone or conjugated for inducing an immunological response.

The present invention provides the use of the above composition wherein the peptide is alone or conjugated for the production of antibodies.

The present invention provides a kit for the diagnosis of a tumor comprising the peptide alone or conjugated or the composition comprising the peptide alone or conjugated.

In the present invention, peptide derivatives are also contemplated. A derivative of the peptide which includes the sequence AVCGTG (SEQ ID NO. 1) is a peptide obtained by technologies known in the field. The derivative is a mutant protein that differs from the amino acid sequence AVCGTG (SEQ ID NO. 1) by the mutation of a single or more amino acids. In a preferred embodiment of the present invention, only one amino acid substitution is inserted in the sequence of the native protein. However they are included in the essence of the present invention of the protein AVCGTG (SEQ ID NO. 1) that can be further optimized by replacement of a plurality (for example two or more) of amino acid substitutions. The derivatives retain the functional properties of the protein sequence AVCGTG (SEQ ID NO. 1), in particular the ability to act as a carrier (ability to penetrate into a target site such as a cell or entire organ) and favoring the production of an antibody when combined to an antigen. The ability to act as a carrier can be measured as described in materials and methods section. Other methods to measure the ability to act as a carrier are described in the technical section and are suitable.

The terms “mutation” or “derivative” or “variant”, as used in the context of the present invention can be interpreted as substitution, deletion and/or addition of single amino acid in the target sequence. Preferably, the mutation of the target sequence in the present invention is a substitution. The replacement can be done with a genetically encoded amino acid or an amino acid not genetically encoded. Examples of non-genetically encoded amino acids are homocysteine, hydroxyproline, omithin, hydroxylysine, citrulline, carnitine, etc.

A further aspect of the present invention is a pharmaceutical composition which includes the peptide with the following sequence AVCGTG (SEQ ID NO. 1), a derivative or a biologically active fragment as an active ingredient. The pharmaceutical composition of the present invention can be used for diagnostic or for therapeutic applications.

The exact formulation, ways of administering and dosage can be chosen by the individual G.P. according to the patient's condition. The dose can be given once or repeatedly, at intervals. Dosage amount and interval may be adjusted individually to provide the therapeutic and/or diagnostic effect, which results in the decrease of symptoms or a longer survival of the patient.

The actual amount of composition given for treatment or diagnostic purposes, will, of course, depend on the patient's conditions, his weight, the severity of illness, the ways of administering and the opinion of the prescribing physician. A suitable daily dosage will range from 0.001 to 10 mg/kg, in particular 0.1 to 5 mg/kg. The administering may be carried out by well known methods, for example: by injection, in particular intravenous, intramuscular, transmucosal, subcutaneous or intraperitoneal injection and/or oral, topical, nasal, inhalation, aerosol and/or rectal application, etc. The administering may be local or systemic. Also, the peptide which includes the sequence AVCGTG (SEQ ID NO. 1), ortholog, derivative or its biologically active fragment, essence of the present invention, can be reversibly immobilized and/or adsorbed on the surface and/or inside medical devices or release systems/transmission of drugs (microspheres). Medical devices and microspheres can be reversibly loaded with the peptide which includes the sequence AVCGTG (SEQ ID NO. 1), derivative or a biologically active fragment of this invention, through their binding, impregnation and/or adsorption on the surface of the medical device or of the microsphere or on a level which covers the surface. When the medical device or the microspheres are in contact with the biological fluids, the peptide with the following sequence AVCGTG (SEQ ID NO. 1), a derivative or a biologically active fragment of it reversibly immobilized is released. Therefore, the medical device and the microsphere act as tools that elute the molecule essence of the present invention in such a way that their release kinetics can be controlled, ensuring sustained release, as required by the pharmacological treatment of release. The methods for coating/impregnating the medical devices and loading microspheres are well known by experts in these technologies.

The essence of the invention is a derivative, an equivalent, and a fragment of a peptide consisting of the sequence AVCGTG (SEQ ID NO. 1).

As used herein, the word “equivalent” means a peptide having at least one of the activities of the peptide with the following sequence AVCGTG (SEQ ID NO. 1). “Homologus” shall mean a peptide that features some changes compared to the peptide with the following sequence AVCGTG (SEQ ID NO. 1). These changes may be a deletion, a truncation, an extension, a chimeric fusion, and/or a mutation. Among the equivalent peptides, the ones that show more than 80% homology are preferred.

“Derivative” refers to any peptide, possibly mutated, truncated, and/or extended, which have been chemically modified or contain unusual amino acids. The peptide of the invention, if required, can be modified in vitro and/or in vivo, for example by glycosylation, myristoylation, amidation, carboxylation or phosphorylation, and may be obtained, for example, by synthetic or recombinant techniques known in the field.

As used herein, the term “orthologs” refers to proteins in different species than the proteins SEQ ID NO. 1 in Homo sapiens. As an example of such orthologs, one can quote the corresponding proteins in AVCGTG (SEQ ID NO. 1) in Mus musculus, Rattus norvegicus, Gallus gallus, Xenopus laevis and Daino rerio.

As used herein, the term “derivatives” refers to peptides having a percentage of identity at least of 75% with SEQ ID NO. 1, preferably at least of 85%, for example at least of 90%, and more preferably at least of 95%.

In the present invention, the term “fragments” refers to peptides having a length of at least 5 amino acids, preferably at least 4 amino acids, as an example of at least 3 amino acids. In the present invention, all of the fragments and derivatives possess the ability of penetrating cells. The composition of the invention contains also excipients or pharmaceutically acceptable excipients. The term excipients “pharmaceutically acceptable” or “acceptable” refers to molecular entities and compositions that are physiologically tolerable and do not produce a typical allergic reaction such as gastric upset, dizziness and such, when administered to a human being. Preferably, as used herein, the term “pharmaceutically acceptable” means approved by a regulatory agency. The term “excipient” refers to a diluent, adjuvant, or transmitter with which the compound is administered. Such pharmaceutical carriers can be sterile liquids, such as water and oils, including those of petroleum, animal, and vegetable or of synthetic origin, such as peanut oil, soybean oil, mineral oil, sesame oil and such. Salt solutions of water or an aqueous solution and aqueous dextrose and glycerol solutions are preferably employed as carriers, particularly for injectable solutions. Suitable pharmaceutical carriers are described in “Remington's Pharmaceutical Sciences” by EW Martin.

As used herein, the term “nucleotide” refers to RNA or DNA, preferably to DNA. Given DNA may be double-stranded or single-stranded.

Preferably, the nucleotide includes a sequence that encodes the sequence of the peptide by the following sequence AVCGTG (SEQ ID NO. 1).

The nucleotide of the invention might also include the encoding sequence of the peptide previously described, and additional encoding sequence as leader sequence or a pro-protein sequence, and/or additional non-coding sequences, such as sequences UTR.

As used herein, the term “vector” refers to an expressing vector and may for example be in the form of a plasmid, a viral particle, a phage, etc. Such vectors may include bacterial plasmids, phage DNA, baculovirus, yeast plasmids, vectors derived from combinations of plasmids and phage DNA, viral DNA such as vaccinia, adenovirus, pox birds, pseudo-rabies. A large number of suitable vectors are known to the experts in the field and are available on the market. The following vectors are provided as instances. Bacterial: pQE70, pQE60, pQE-9 (QIAGEN), PBS, pDIO, phagescript, psiX174, pBluescript SK, pbsks, pNH8A, pNH1 [beta] a, pNH18A, pNH46A (STRATAGENE), ptrc99a, pKK223-3, pKK233-3, pDR540, pRIT5 (Pharmacia). Eukaryotes: pWLNEO, pSV2CAT, pOG44, pXT1, PSG (STRATAGENE), pSVK3, pBPV, pMSG, pSVL (Pharmacia). However, any other vector may be used as long as it is replicable and viable in the host. The polynucleotide sequence, preferably the DNA sequence in the vector is operatively linked to a control sequence of appropriate expression (promoter) for a direct synthesis of mRNA. As typical examples of such promoters, we could mention a prokaryotic or eukaryotic promoter such as CMV immediate early, HSV thymidine kinase, early and late SV40, LTR retrovirus. The expression vector also contains a ribosome binding site for translation initiation and a transcription vector. The vector may also include appropriate sequences for amplifying expression.

Furthermore, the vectors preferably contain one or more selectable marker genes able to provide a phenotypic trait for selection of transformed host cells such as di-hydro folate reductase or neomycin resistance for eukaryotic cell culture, or such as tetracycline or ampicillin resistance in E. coli.

As used herein, the term “host cell genetically engineered” relates to host cells that have been transduced, transformed or transfected with the nucleotide or with the vector previously described.

As typical examples of appropriate host cells, we could mention bacterial cells, such as E. coli, Streptomyces, Salmonella typhimurium, fungal cells such as yeast, insect cells such as Sf9, animal cells such as CHO or COS, plant cells, etc., the selection of an appropriate host is considered by experts in the field of application.

Preferably, the above mentioned host cell is an animal cell, and more preferably a human cell. The introduction of nucleotide or of the vector previously described into the host cell can be effected by well-known method by an expert in the field, such as transfection with calcium phosphate, DEAE-dextran mediated transfection, or electroporation.

The composition of the invention may include one or more additives (for example, stabilizers, preservatives).

According to the present invention, an “effective amount” of a composition or of the peptide of the invention is an amount that is sufficient to achieve the desired biological effect, in this case of penetrating the cells and releasing in them what has been chemically combined. It is understood that the effective dosage will be dependent on age, sex, health and weight of the patient, kind of concurrent treatment (if any), frequency of treatment, and the nature of the desired effect. The ranges of effective doses provided below are not intended to limit the invention and represent intervals of preferred doses. However, the preferred dosage can be tailored to the individual patient, as understood and determinable by an expert of the field, without undue experimentation. Such peptide, nucleotide, vector, and host cell are as described previously.

In the present invention, a carrier molecule (such as the peptide of the invention) is a molecule capable of transporting ions and organic molecules through cell membranes and generally requires a protein carrier (carrier). Because the ions and molecules that must permeate are often too polar or too large to penetrate into the membranes alone, they require a carrier. There are many examples of such carriers, including those responsible for the transport of glucose and amino acids into the cells, the transport of sodium ions and calcium from the inside to the outside of the cells, the absorption in the nerve endings of the precursors of neurotransmitters (such as choline) or of neurotransmitters themselves (as in the case of noradrenaline, 5-HT, the glutamate, and peptides). Transporter proteins contain a recognition site, which defines them as specific for a permanent species, and it is not surprising that these recognition sites can also be targets for drugs, aiming to block the transport system. In the present invention, the peptide can be combined to molecules with therapeutic activity, cytostatic, metals, enzymes, radioactive isotopes issuers radiation or β/γ, molecules with antigenic activity or antibody molecules.

The peptide of the invention alone or conjugated to a molecule may also be administered in combination with other therapeutic agents such as chemeterapeutic agents, antibiotics ect. The combination may be simultaneous or sequential.

The present invention will be illustrated with non-limiting examples in reference to the following figures.

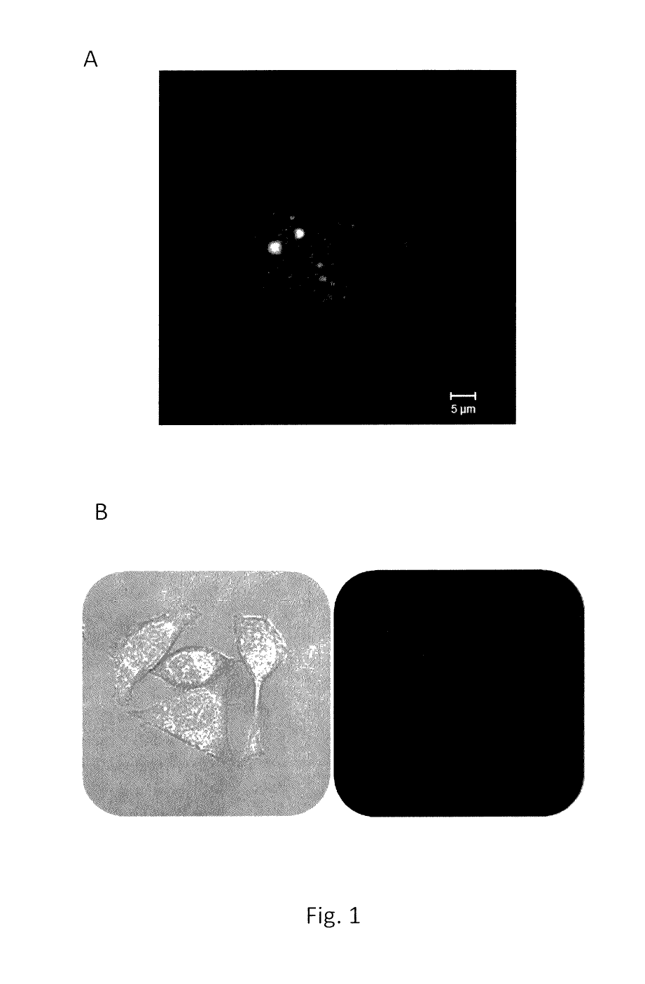

FIG. 1: (A) Confocal picture of transmission and fluorescence of cells of human breast cancer (MCF-7) incubated for 3 hr., In the presence of the fluoresceinated hexapeptide (FITC-PEP). The image clearly indicates that the fluoresceinated peptide was internalized into the cytoplasm of target cells. (Exc. 488 nm Argon Laser/em. BP500-550 filter), (B) The image in Fluorescence Optical Microscopy shows that the synthetic sequence of minipeptide conjugated with fluorescein (FAM 5.6) penetrates into the MCF-7 breast cancer cells spreading in their cytoplasme.

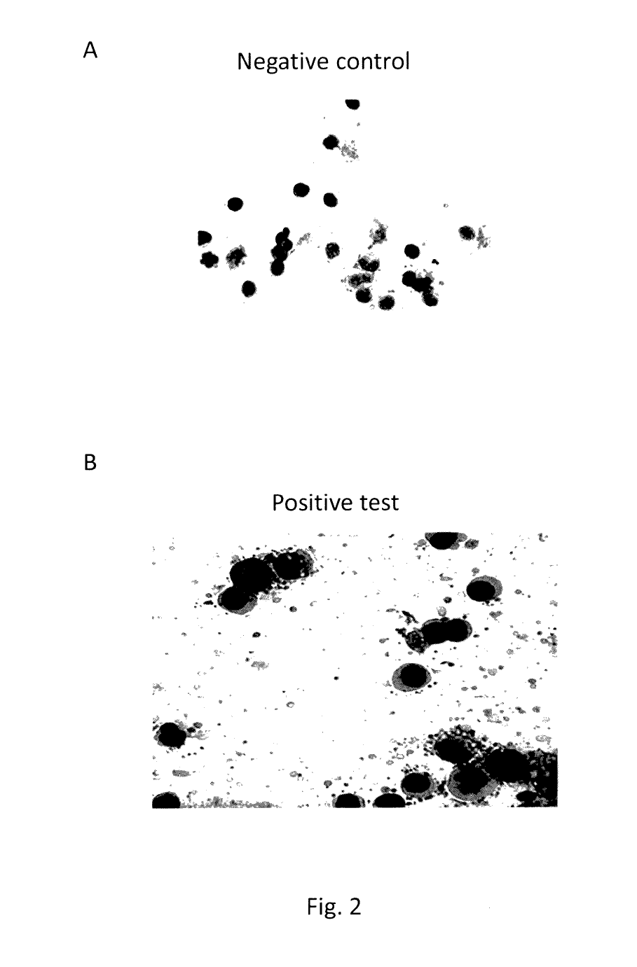

FIG. 2: Optical microscope image of examination in immunologic cyto-chemistry of white blood cells to which the hexapeptide was added. The positive reaction indicates that the peptide has penetrated into the cells, prompting that when combined to a substance, it is deposited within the cell.

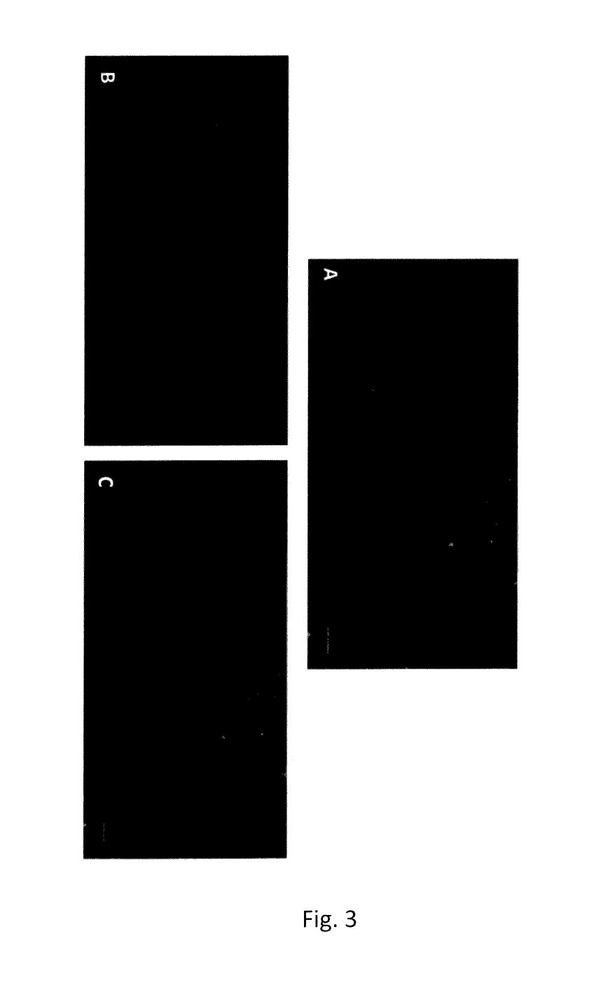

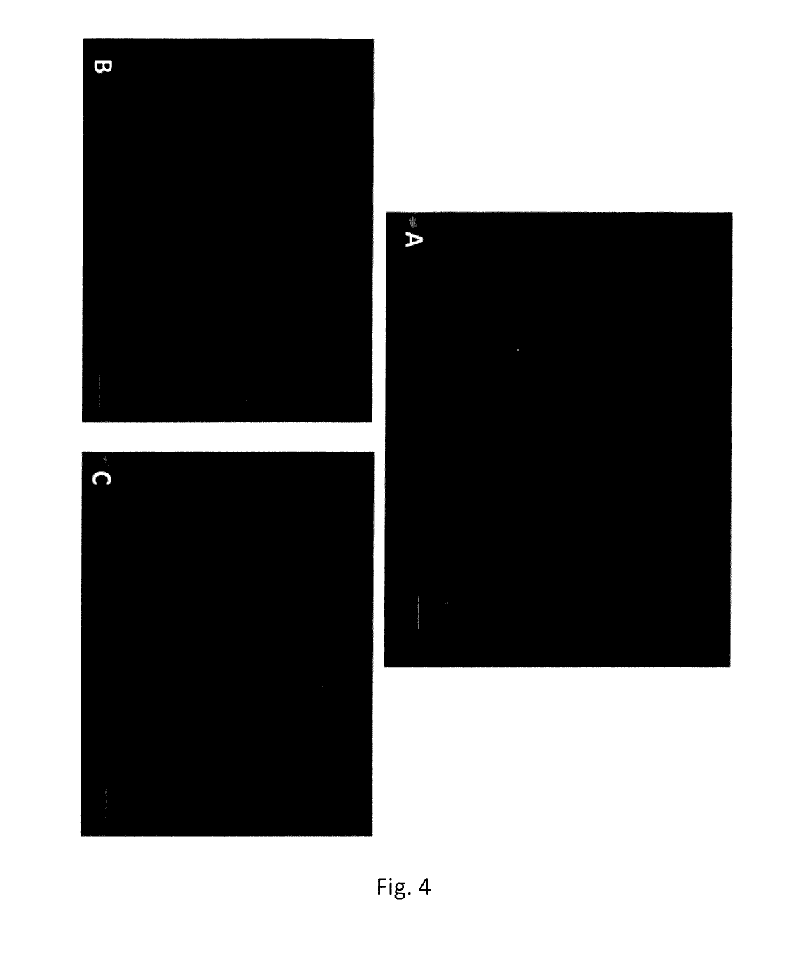

FIGS. 3-4: hexapeptide immunofluorescent detection in lymphocytes from healthy pediatric patient. Lymphocytes of healthy pediatric patient following 3 hour incubation with hexapaptide Green fluorescent secondary antibody reveals the cytoplasmic internalization of hexapeptide. A) Merge of B) and C) images. B) Nuclei stained in blue (DAPI) and C) cytoplasmic green fluorescence. SCALE BAR: 3 μm

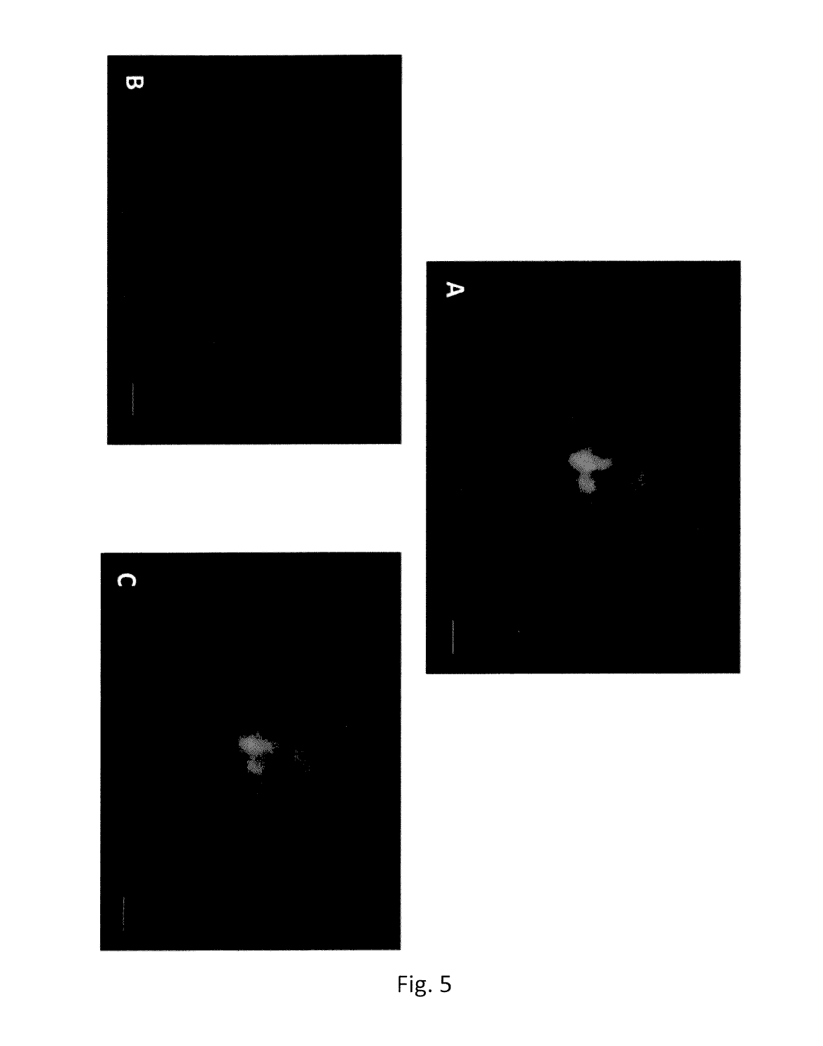

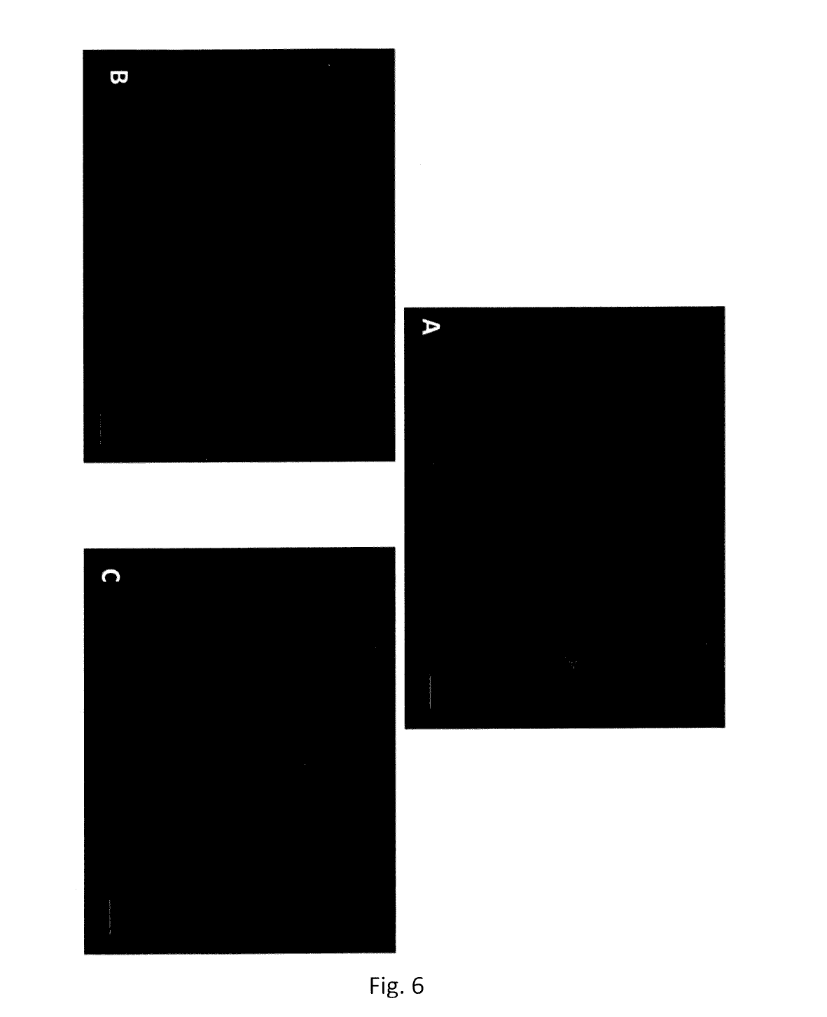

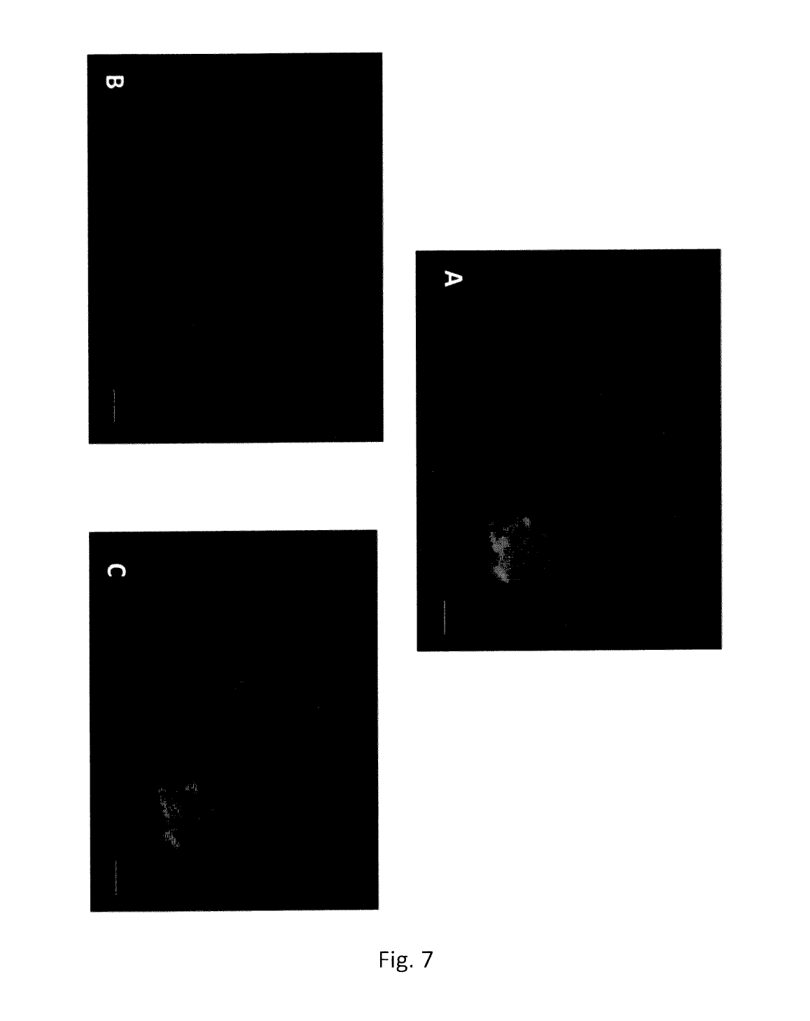

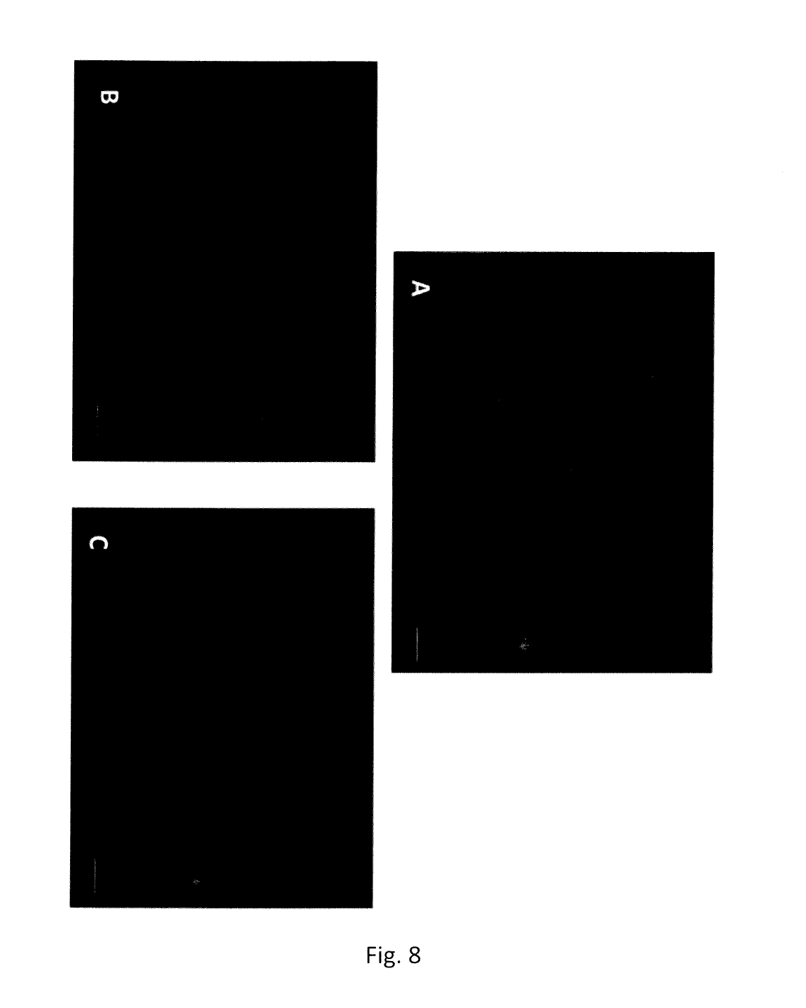

FIGS. 5-8: hexapeptide immunofluorescent detection in Jurkat cells. Jurkat cells following 3 hour incubation with hexapeptide Green fluorescent secondary antibody reveals the cytoplasmic internalization of hexapeptide. A) Merge of B) and C) images. B) Nuclei stained in blue (DAPI) and C) cytoplasmic green fluorescence. SCALE BAR: 4 μm

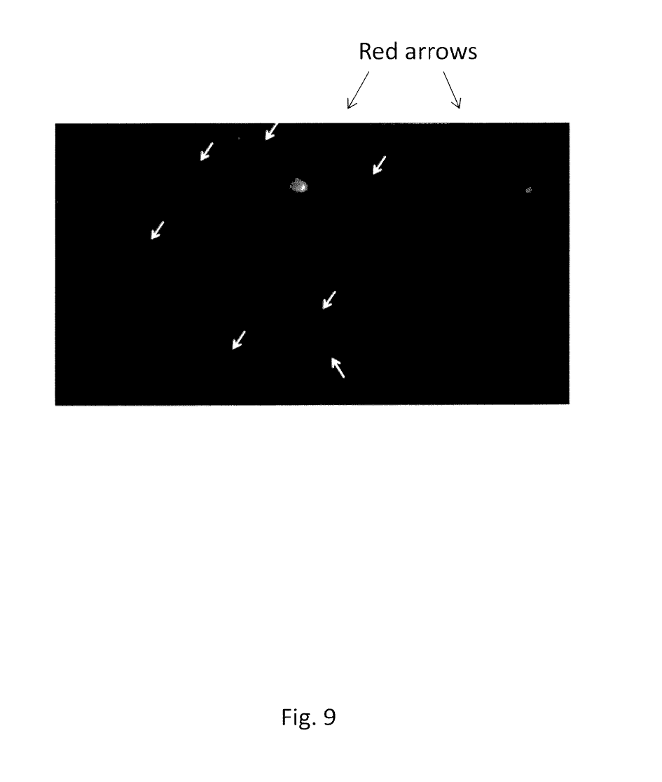

FIG. 9: Sperm fluorescence microscopy. The red arrows indicate sperm that do not have an intact membrane, therefore propidium iodide penetrates into and intercalate into DNA by emitting red fluorescence. The white arrows indicate sperm that do not exhibit red fluorescence (so that they have a membrane intact spermatozoa because vital and functional) and have a green fluorescence localized in the mid piece of the tail, the same region in which mitochondria reside.

### DETAILED DESCRIPTION OF THE INVENTION

### Example 1

### Hexapeptide Sequence, Synthesis, Combination

### Synthesis

Peptide (also named in the description as minipeptide or hexapeptide-rMnSOD-Lp) was synthesized step-wise in batch-mode on solid support with automatic synthesizer Syro (MultiSynTech), using Fmoc/tBu chemistry [12] starting from the C-terminal residue pre-loaded on PS-PHB resin with an average of 0.57 mmol/g substitution (Rapp Polymere). Synthesis on a 30 micromol scale proceeded through standard cycles of Fmoc deprotection [piperidine:DMF, 0.2:1(v/v)] and Fmoc-aminoacid coupling steps [Resin:Fmoc-aminoacid:HBTU:DIEA, 1:4:4:8] [13].

As Pt binder, diamino-ethyl-glycine was used which, by virtue of the presence of 2 free amine groups is able to combine platinum(II) ions as PtCl2 [16].

### Amino Acid Derivatives

Fmoc-Ala-OH, Fmoc-Val-OH, Fmoc-Cys(Trt)-OH, Fmoc-Gly-OH, Fmoc-Thr(tBu)-OH.

### Binder

N-Fmoc[N′-Fmoc-(2′amino ethyl)]glycine.

### Cleavage

Side chain de-protection with concomitant cleavage of peptide from solid support was achieved suspending the protected peptide-resin in a mixture TFA:water [95:5 (v/v)] for three hours. The resin was removed by filtration under reduced pressure. Precipitation was achieved collecting the filtrate on a bed of cold ether. After several ether washes, crude compound was suspended in water (0.1% TFA, v/v) and lyophilized.

### Analysis

Purity was assessed by analytical RP-HPLC (Shimadzu): Vydac C18 column (4.6×150 mm); Eluent A: 0.1% TFA in water; Eluent B: 0.1% TFA in acetonitrile; gradient: from 5% B to 65% B in 20 min; flow 1 ml/min. (R.t.): 16.5 minutes.

Identity was assessed by mass spectrometry performed on a MALDI-Tof spectrometer (Applied BioSystem) [MH+=870].

### Purification

RP-HPLC purification was performed on either Vydac C18 column (22×250 mm); eluent A: 0.1% TFA in water; eluent B: 0.1% TFA in acetonitrile; gradient: from 5% B to 65% B in 40 min; flow 20 ml/min).

### Example 2

### Hexapeptide Ability to Penetrate into the Cells. Assessment by Confocal Microscopy

### Materials and Methods

### Confocal Analysis

The confocal analysis was performed by placing the cells of human breast cancer (MCF-7, ATCC HTB-22) on suitable slides (MatTek, Ashland Corporation MA, U.S.). The hexapaptide to the concentration of 0.125 μM was then added to the cells.

### Hexapeptide Combination to Fluorescein

### Synthesis

Peptide was synthesized step-wise in batch-mode on solid support with automatic synthesizer Syro (MultiSynTech), using Fmoc/tBu chemistry [1] starting from the C-terminal residue pre-loaded on PS-PHB resin with an average of 0.57 mmol/g substitution (Rapp Polymere). Synthesis on a 30 micromol scale proceeded through standard cycles of Fmoc deprotection [piperidine:DMF, 0.2:1(v/v)] and Fmoc-aminoacid coupling steps [Resin:Fmoc-aminoacid:HBTU:DIEA, 1:4:4:8] [2].

### Amino Acid Derivatives

Fmoc-Ala-OH, Fmoc-Val-OH, Fmoc-Cys(Trt)-OH, Fmoc-Gly-OH, Fmoc-Thr(tBu)-OH. Fluorescein was coupled to peptidil-resin using 5(6)-FAM:HBTU:DIEA in ratio 4:4:8 respect peptidyl-resin.

### Cleavage

Side chain de-protection with concomitant cleavage of peptide from solid support was achieved suspending the protected peptide-resin in a mixture TFA:water [95:5 (v/v)] for three hours. The resin was removed by filtration under reduced pressure. Precipitation was achieved collecting the filtrate on a bed of cold ether. After several ether washes, crude compound was suspended in water (0.1% TFA, v/v) and lyophilized.

### Analysis

Purity was assessed by analytical RP-HPLC (Shimadzu):

Vydac C18 column (4.6×150 mm); Eluent A: 0.1% TFA in water; Eluent B: 0.1% TFA in acetonitrile; gradient: from 5% B to 65% B in 20 min; flow 1 ml/min. (R.t.): 16.5 minutes Identity was assessed by mass spectrometry performed on a MALDI-Tof spectrometer (Applied BioSystem) [MH+=866].

### Immunocytochemistry

The protocol used for the internalization of the PEP-6 FAM includes two separate experiments. A first experiment was done using PEP 6-FAM to 92 μM for 3 hr and the internalization was assessed. To these cells, already treated with PEP6-FAM, primary Ab anti-PEP 24 1:20 overnight at 4° C. was added and, as secondary antibody goat anti rabbit fluorescent CF 488A; and to 1 plate, already treated with PEP6 FAM, it was added the primary antibody anti rMnSOD 1:20 overnight and the secondary CF555 rodamynated donkey anti sheep. For 45 min-1 hr.

In the second experiment, the PEP 6-FAM was used at 92 μM to 1 hr and 3 hr with fixation in ethanol and paraformaldehyde, and for both times it was assessed the penetration alone, without the addition of the antibody and with the addition of primary antibody anti rMnSOD with three different dilutions (1500-1:1000-1:2000) in 5% NGS (Normal Goat Serum) in PBS +0.1% Triton overnight at 4° C. and, the secondary antibody anti rhodium CF 555 sheep 1:400 for 45′-1 hr. The nuclei were subsequently stained with 10 μg for ml⁻¹ DAPI (4′,6-diamidin-2-phenylindole, LIFE™ Thencologies). The slides were then put together and stored at 4° C. until the examination was achieved at the LSM 510 META confocal microscope, Zeiss (10).

### Results

The analysis performed by confocal microscopy clearly showed that the fluorescent peptide is internalized in the cytoplasm of MCF-7 cells (FIG. 1A). The image (by transmission and fluorescence) of MCF-7 cells incubated with 0.125 μM of FTC-peptide for 3 hr., indicates the internalization of the peptide (exc. 488 nm Argon Laser/em. BP500-550 filter). The image in fluorescence optical microscopy shows how the synthetic sequence of minipeptide combined to fluorescein (5.6 FAM) penetrates into the MCF-7 breast cancer cells spreading in their cytoplasm (FIG. 1B).

### Example 3

### Ability of the Hexapeptide to Transport into the Cells a Conjugated Molecule. Hexapeptide Conjugated to Cisplatin (Cisplatin-AVCGTG)

### Materials and Methods

Samples of each cell line: PA-TU 8902 Pancreatic Adenocarcinoma (DSMZ No. ACC179), A375 melanoma (ATCC CRL 1619™) Prostate Cancer DU145 (ATCC HTB 81), HUH-7 Hepatocarcinoma JCRB Cell Bank (JCRB043 NIBIO, National Institute of Biomedical Innovation), A2780 Ovarian Cancer (European Collection of Cell Cultures, ECACC, Salisbury, Wiltshire, UK), MRC-5 Human Lung Fibroblasts Normal (ATCC CCL-171™) Breast cancer MCF-7 (ATCC HTB-22™) used in the study were treated, independently, for 3 hours with a solution containing 92 μM of cisplatin alone (CC, Bristol-Mayer Co. (NewYork City, United States) or with a solution of 92 μM of leader-hexapeptide rMnSOD-Lp-CC (hex-Lp-CC) containing 11.1 μg of cisplatin. The cells were maintained in DMEM culture medium added with 5% fetal bovine serum (FCS) (GIBCO), and the controls were prepared by maintaining the cells in the same culture conditions but in the presence of a solution of 92 μM of the peptide only, therefore in the absence of cisplatin. The cells to be examined, after the incubation period, were harvested, washed twice in buffered solution (PBS) and treated with 50 μl of 35% HNO₃ for 16 hours. The platinum (Pt) contained in the culture medium or in the cell pellet was determined by spectrophotometer at atomic absorption (Analyst 800, Perkin-Elmer, Norwalk, Conn.) using the following criteria: temperature of pretreatment, 1300° C., temperature of atomizing 2200° C. 0.015 μg of palladium were used (palladium has been used for the modification of the matrix) and 0.01 μg of Mg (NO₃)₂ as a modifier of the matrix. The determinations were performed using a graphite furnace equipped with the correction system of the Zeeman effect. For the determination of the metal, tubes of pyrolytic graphite coated with THGA were used (Perkin-Elmer) with an integrated “vov-type platform” system. A standard solution of platinum in 2.5% HNO₃ (Spectrascan) was used as a mother solution for the construction of a calibration curve with three points of reference. Each determination was performed independently, in triplicate (11).

### Results

### Spectrophotometric Analysis in Atomic Absorption

The ability of the peptide to penetrate into tumor cells and to release cisplatin was assessed on 5 cell lines of human cancers (Breast, Ovarian, Hepatocellular carcinoma, renal cell carcinoma, melanoma) by atomic absorption spectrophotometry. The results of the analysis are shown in Table I.

TABLE I
Determination of platinum in the supernatant and in the cell
pellet of different cell lines of human tumors treated with
Lp-CC or CC. The concentration of platinum (Pt) was assessed
by spectrophotometer analysis in atomic absorption.

| | CC treatment | | | Treatment Lp-CC | | |
|---|---|---|---|---|---|---|
| | Pt in the | | | Pt in the | | |
| | culture | Pt cell | Cellular | culture | Pt cell | Cellular |
| | medium | pellet | uptake | medium | pellet | uptake |
| Sample | (μg/L) | (μg/L) | of Pt (%) | (μg/L) | (μg/L) | of Pt (%) |
| MCF-7 | 29200 | 760 | 2.6 | 2257 | 97 | 4.2 |
| A2780 | 21715 | 163 | 0.75 | 2437.5 | 144 | 5.9 |
| HUH-7 | 18885 | 242.9 | 1.2 | 2257 | 118.7 | 5.2 |
| A375 | 22095 | 399.9 | 1.5 | 2400 | 372 | 15.5 |
| DU145 | 18455 | 167.3 | 0.9 | 2476 | 342.8 | 13.8 |

It is evident that the hexapeptide manages to transport directly inside the cancer cells an amount of platinum that is from 1.6 to 15.3 times higher than the amount of platinum that enters the cells when the Cisplatin is used alone.

After adding to the cells an amount of hexapeptide conjugated to cisplatin, the inventors have found that this synthetic construct had penetrated into tumor cells releasing an amount of platinum that percentage wise is significantly greater than the amount of platinum that reaches cancer cells when cisplatin is used alone. In terms of %, the uptake by tumor cells varies from cell to cell and ranges from 4.2 to 15.5%. By contrast, the use of the same amount of Cisplatin, alone, has shown that if it is not conjugated to the carrier peptide, the quantity of cisplatin that will be found within the cells will range from 0,7% and 2,6%. The solutions of Cisplatin and Peptide-conjugated Cisplatin that were used have the same molarity which is equal to 92 μM and both contain 11.1 μg of Cisplatin.

### Example 4

### Immuno-Histochemical Assessment of the Internalization of the Hexapeptide into Tumor Cells of Human Origin—Assessment Performed Qualitatively by Optical Microscope

### Materials and Methods

### Immunocytochemistry

The tumor cells were incubated for 3 hours in the presence or absence of hexapeptide conjugated to cisplatin (hex-Lp-CC), or cisplatin alone (CC) at concentrations of 92 μM. Subsequently, a cytospin was used to obtain a smear of cells on slides. The cells were then placed in Zamboni solution (4% paraformaldehyde, 15% picric acid) for 60 min and then washed with a solution of 1×PBS and incubated for 5 min with 3% hydrogen peroxide to suppress the action of endogenous peroxidase. The immuno-coloring was performed using the Dako LSA+System HRP kit (DAKO, CA, USA). The incubation lasted 30 minutes with primary antibody anti-peptide leader of 24 aa (1:200), produced in rabbits. This phase was followed by sequential method that requires a 30-min incubation with biotinylated antibody and peroxidase-labeled streptavidin. To complete the reaction of differentiation, a substrate-chromogen solution was engaged. Cell smears were colored with hematoxylin.

### Results

The results of the analysis are shown in Table II.

TABLE II
Immuno-histochemical assessment of the internalization
of the hexapeptide into tumor cells of human origin

| | | Cells treated with | | |
|---|---|---|---|---|
| | | hexapeptide + | Control | |
| | | Cisplatin | only | |
| | Cells | (for 3 hours) and | with | Cells with |
| | without | examined with | Ab-anti- | rMnSOd + |
| | any | Ab anti-Lp. | peptide | Ab. Anti-pep. |
| Cell line | treatment | (24 aa) | (24aa) | (24 aa). |
| Human breast | negative | positive | negative | positive |
| cancer MCF-7 | | | | |
| A-2780 | negative | positive | negative | positive |
| Ovarian Ca | | | | |
| HUH HCC | negative | positive | negative | positive |
| A-375 | negative | positive | negative | positive |
| Melanoma | | | | |
| DU 145 | negative | positive | negative | positive |
| Renal Cell | | | | |
| Carcinoma | | | | |

A positive reaction is represented by a brownish coloration. A blue coloration indicates negativity.

The immunocytochemical analysis was performed using a polyclonal antibody able to recognize the amino acid sequence of the peptide leader of rMnSOD (7), ie of 24 aa. among which there are also those that form the hexapeptide.

The target cells were treated according to the following scheme:
Cells that have not undergone any treatment and used as a negative control,
Cells that were treated in the presence of hexapeptide conjugated to cisplatin,
Cells that have been treated with the antibody alone,
Cells that have received the entire molecule rMnSOD bearing, as previously mentioned, the hexapeptide sequence included in the sequence of the leader peptide of 24 aa.

After incubation with these molecules, the cells were washed and treated with the polyclonal antibody for three hours at rt, and then the cells were washed with a solution of PBS [17, 18]. In control cells (not treated and those treated with the antibody alone), the reaction is negative. In cells treated with the hexapeptide (alone) or the entire protein that bears, within its leader sequence of 24 aa the six amino acids that compose the hexapeptide, the reaction was positive because the antibody has recognized the amino acids present in this sequence. The presence of the hexapeptide recognized by the antibody is represented by a brownish coloration, such as that represented in the monocytes (FIG. 2B).

### Example 5

### Assessment of the Ability of the Hexapeptide Conjugated to a Chromophore Group (Fluorescein) to Enter Cells

The test results are positive and confirms the previous data. That is, the hexapeptide, bounded (conjugated) with fluorescein before the execution of the immunocytochemistry examination, penetrates into the cells.

Therefore, the hexapeptide can be used to carry any molecule in the cells for diagnostic or therapeutic purpose, or to trigger an immune response.

A peptide consisting of similar amino acids but with “scrambled” sequence (ACTGVG, SEQ ID NO. 2) gave negative results, that is, it does not penetrate into the cells.

### Example 6

### Cytotoxicity of the Hexapeptide Conjugated to Cisplatin

### Material and Methods

The leader peptide of 24 aa. (rMnSOD-Lp), alone, or conjugated to cisplatin (rMnSOD-Lp-CC), the hexapeptide AVCGTG (SEQ ID NO. 1) alone (hex-Lp), or conjugated to cisplatin (hex-Lp-CC) and finally the cisplatin alone (CC) were added to the target cells (MCF-7, A-2780; HUH-7; MRC-5; DU-145) at a concentration of 1,5 μM. The release of lactate dehydrogenase (LDH) was then measured quantitatively in the supernatant of the culture medium of the cells after 24 hr of incubation, using the Cytotox 96 non riadioactive cytotoxic assay kit in compliance with the technical indications provided by Promega. The experiments were performed in triplicate in an independent manner, using different preparations. (Table III)

### Results

The results of the analysis are shown in Table III.

TABLE III
Cytotoxicity of hexapeptide conjugated to cisplatin

| | | Hexapeptide + | |
|---|---|---|---|
| | Only Cisplatin | Cisplatin | Hexapeptide |
| | 1.5 μM | 1.5 μM | 1.5 mM |
| Cell line | LDH release | LDH release | LDH μelease |
| Human breast | 48% | 77% | 5% |
| carcinoma MCF-7 | | | |
| A-2780 | 36% | 85% | 5% |
| PaTu Ca pancreatic | 25% | 60% | 3% |
| MRC-5 | 37% | 40% | 2% |
| HUT-7 | 32% | 69% | 4% |
| Hepatocarcinoma | | | |

Cisplatin alone (CC), the hexapeptide conjugated to cisplatin (hex-CC) and the hexapeptide alone (hexapeptide) were added to the target cells at a concentration of 1.5 μM and maintained for 48 hr. After the incubation the amount of lactate dehydrogenase (LDH) released from cells into the supernatant was measured. This quantity was expressed as a percentage of the total LDH (released by cells in which cell lysis had been induced using a detergent). Approximately 3% of LDH was found in the culture medium of not treated cells.

The synthetic construct consisting of the hexapeptide conjugated to cisplatin, added to the tumor cells has penetrated into the cells, releasing an amount of cisplatin that has caused their immediate death. The assessment of the induced cytotoxicity was performed using the enzyme lactate dehydrogenase and expressed in terms of %.

As can be seen from the results shown in Table III, the hexapeptide alone is practically devoid of toxicity, both on tumor and normal cells (MRC-5). In fact, the amount of LDH release (LDH is an enzyme that reflects the degree of cell mortality) is very low, ranging from 2% to 5%. On the contrary, when the hexapeptide conjugated to cisplatin is added to the cells, the cell mortality increases significantly ranging from 40% to 85%.

Cisplatin alone, instead causes a mortality that varies from a minimum of 25% to a maximum of 48%, that is twice or three times less than the one induced by the hexapaptide combined to cisplatin.

The experiment clearly indicates that the hexapeptide transports and releases a double or triple amount of cytostatic directly into the cells. Therefore, the therapeutic index of hexapeptide increased significantly. The peptide has greater efficacy and less side effects.

### Example 7

### White Blood Cell Penetration

### Material and Methods

### Culture Cells

Human Normal Lymphocytes cells were collected from healthy donors and purified using Ficoll Hystopaque density gradient centrifugation. The cells were cultured at a density of 1×10⁶ cells/mL in RPMI media supplemented with 1% Penstrep and 10% FBS in a humidified atmosphere containing 5% CO₂ at 37° C.

### Immunocytochemistry at Light Microscopy

Lymphocytes were incubated for five hours in the presence of fluorescinated Hexapeptide 92 μM. A cytospin was used to smear cells onto slides. The smears were fixed in Zamboni solution (4% paraformaldehyde, 15% picric acid) for 60 minutes and then washed with 1×PBS and incubated for five minutes with 3% hydrogen peroxide to quench endogenous peroxidase activity. Immunostaining was performed using the DAKO LSA+System HRP kit. Rabbit anti-rMnSOD-Lp (1:200), was incubated on slides for 30 minutes, followed by incubation with the biotinylated secondary antibody for 30 minutes and peroxidase-labeled streptavidin for 30 minutes. To complete the reaction, a substrate-chromogen solution was used. The smears were counterstained with hematoxylin.

### Light Microscopy

The treatments performed respectively with 92 μM AVCGTG (SEQ ID NO. 1)-FITC for 3 hours on the three cell populations gave the following percentages of labeled cells: 95% of lymphocytes, while the controls displayed no labeling (FIG. 3,4). Overall, these results indicate that most cells internalized AVCGTG (SEQ ID NO. 1)-FITC, as they displayed both mainly cytoplasmic and less nuclear immunoreactivity (FIG. 3,4).

It is observed that the fluorescent hexapeptide of the invention constituted of 6 aa. penetrates in white blood cells (lymphocytes and neutrophils) of healthy subject. Therefore, the peptide of the invention is particularly useful to target leucocyte cells.

The peptide by being conjugated to a citostatic molecule is advantageous for the treatment of leukemia. Citostatic molecules include Idarubicin, Etoposide or cytarabin. Further the peptide of the invention may also be conjugated to an antibiotic molecule, allowing such molecules to directly target white blood cells where bacteria tend to hide to provoke resistant infectious diseases.

The peptide may also be successfully conjugated to disease specific antigens in order to induce immune response.

### Example 8

### Jurkat Cell Penetration

### Material and Methods

### Immunofluorescence Protocol

Jurkat cells (ATTC, UK) were seeded at density of 150000 cells/well in RPMI media supplemented with 1% Penstrep and 10% FBS in a humidified atmosphere containing 5% CO₂ at 37°. The next day, the cells were incubated with 92 μM AVCGTG (SEQ ID NO. 1)-FITC for 3 h in multiwell 24. Subsequently, a cytospin was used to smear cells onto slides. The smears were fixed with 4% paraformaldehyde/PBS for 10 min and rinsed twice with 1×PBS containing 0.1% Triton to remove traces of fixative. Cell samples without AVCGTG (SEQ ID NO. 1)-FITC were used for the background control. After blocking solution with 5% NGS (Normal Goat Serum, VECTOR) 1×PBS+0,1% Triton X-100, was added for 30 minutes. After removing the blocking solution, cells were incubated with primary antibody hexapeptide, diluted 1:200 in blocking solution, overnight at 4° C. The following day, after washing with lx PBS and 0.1% Triton X-100 for 10 minutes, secondary antibody (ALEXA FLUO 488A, SANTACRUZ) diluted 1:400 with 1×PBS and 0.1% Triton X-100 for 2 h was added. The excess of antibody was removed with 3 washes with 1×PBS.

Nuclei were stained with DAPI (1 μg/mL). The slides were mounted and stored in the dark at 4° C. and then observed by fluorescence microscope ZEISS HB050 by using the software Axion Vision Re1.4.8.

### Results

As shown in FIG. 5-8, the fluorescent hexapeptide of the invention constituted of 6 aa. penetrates in Jurkat cells. Jurkat cells are stabilized human cells deriving from infantile Acute lymphatic leukemia.

The peptide of the invention may be conjugated to a citostatic molecule and is advantageous for the treatment of leukemia. Citostatic molecules include idarubicin, epirubicine or cytarabin. Further, the peptide of the invention may also be conjugated to an antibiotic to target infected organs, leucocytes or bacteria. In particular, the peptide may be conjugated to specific or wide spectrum antibiotics such as β-lactam antibiotics. The peptide of the invention may be advantageously used to render active molecules that became inactive due to bacteria acquired resistance.

### Example 9

### FITC Labeled rMnSOD's Leader Peptide (FITC-rMnSOD-LP)

### Materials and Methods

Three fresh ejaculate from bovine, obtained from two different bulls, were mixed and diluted in Tris solution with a concentration of 100×10⁶ sperm/ml, incubated at 37° C. for 1 hour in the presence of 1 mg/ml of FITC-rMnSOD-LP (hexapeptide) and 1 mg/ml of propidium iodide. Propidium iodide stain is used to identify viable sperm (not red) from the dead ones (red), therefor to prove that sperm are viable. Propidium iodide is a dye that does not cross the cell membrane of a viable sperm, unless this is not broken as a result of sperm death. Taking into consideration only viable sperm (which do not have a red fluorescence), they present a localization of the green fluorescence (hexapeptide) at the level of the mid piece region of the tail, the same location of the mitochondria.

The peptide also enters in the sperm with a non-intact membrane. These sperm must not to be taken into consideration in the penetration study because if the membrane is broken, its permeability is not selective. The hexapeptide enters even in the dead, but for normal diffusion, not for selective activity of intact membranes.

### Results

The 100% of sperm that do not show a red fluorescence (viable sperm), show a green fluorescence localized in the mid piece portion of the tail (FIG. 9). The percentage of positive sperm for propidium iodide varies from 20% to 15%. Therefore the peptide of the invention may be advantageously used to target male gametes. In particular, the peptide of the invention may be linked or conjugated to proteins, nucleic acids, nanoparticles or bioconjugates for therapeutic and diagnostic use for human or animal health.

### REFERENCES

1) Balmain A. (2001) “Cancer genetics: From Boveri and Mendel to microarrays.” Nat. Rev. Cancer 1 (1): 77-82.
2) Bogenman, E. 1996. A metastatic model in SCID mice. Int J. Cancer 67 (3), 379-85
3) Nowak, M A, Komarowa, N L et al. The role of chromosomal instability in tumor initiation. Proc Natl. Acad. Sci USA 99 (25): 1626-31-2002 Dec. 10
4) Duesberg P, Li R Multistep carcinogenesis: a chain reaction of aneuploidizations. Cell Cycle. 2003 May-June; 2 (3):202-10.
5) Loeb L A, Loeb K R, Anderson J P. Multiple mutations and cancer. Proc Natl Acad Sci U.S. A. 2003 February 4; 100 (3):776-81. Epub 2003 Jan. 27.
6) Aldo Mancini, Antonella Borrelli, Antonietta Schiattarella, Stefania Fasano, Antonella Occhiello, et al. Tumor suppressive activity of a variant isoform of manganese superoxide dismutase released by a human liposarcoma cell line. Int J. Cancer: 119, 932-943 (2006)
7) Aldo Mancini, Antonella Borrelli, Antonietta Schiattarella, et al. Biophysical and biochemical characterization of a liposarcoma-derived recombinant MnSOD protein acting as an anticancer agent Int J. Cancer: 123, 2684-2695 (2008).
8) Antonella Borrelli, Antonietta Schiattarella, Roberto Mancini, Franco Morelli, Clemente Capasso, Viviana De Luca, Enrico Gori and Aldo Mancini The leader peptide of a human rec. MnSOD as molecular carrier Which delivers high amounts of Cisplatin into tumor cells inducing a fast apoptosis in vitro Int J. Cancer: 128, 453-459 (2011)
9) J M Stewart, J D Young. Solid phase peptide synthesis. Rockford, Ill.: Pierce Chemical. Cancer 2008; 123:2684-95.,
10) Kubista M, Akerman B, B Norden. Characterization of interaction between DNA and 4′,6-diamidino-2-phenylindole by optical spectroscopy. Biochemistry 26, 4545 (1987)
11) Kowalewska Z, B Welz, I N Castilho, Carasek E. Effect of magnesium acetylacetonate on the signal of organic forms of vanadium in graphite furnace atomic absorption spectrometry. Verlag Chemie, 1983.
12) Atherton E., Sheppard R. C., Solid-Phase peptide synthesis: A practical approach, IRL Press, Oxford University (1989);15.
13) Kates S. A., Albericio F., Carpino L. A., Encyclopedia of Reagents of Organic Synthesis, 4, 2784-2786, Paquette L. A. Ed, Wiley, New York (1996)
14) Borrelli et al. 2010, Int. J. of cancer 128(2):453-459.
15) Borrelli et al. 2012, Chem. Biol. And Drug design 80(1):9-16
16) M. S. Robillard, A. R. P. M. Valentijn, N. J. Meeuwenoord, G. A. van der Marel, J. H. van Boom and J. Reedijk. Angew. Chem. Int. Ed., 39 (2000), p. 3096
17) Hsu S M, Raine L, Fanger H. Use of avidin-biotin-peroxidase complex (ABC) in immunoperoxidase techniques: a comparison between ABC and unlabeled antibody (PAP) procedures. J Histochem Cytochem, 1981; 29:577-80
18) Warnke R, Levy R. (1980)Detection of T and B cell antigens with hybridoma monoclonal antibodies: a biotin-avidin-horseradish peroxidase method. J Histochem Cytochem 1980; 28:771-776.

1- A peptide consisting of the sequence AVCGTG (SEQ ID No. 1).

2- The peptide according to claim 1 being conjugated to a molecule.

3- The peptide according to claim 2 wherein the molecule is selected from the group consisting of: a therapeutic agent, a dye, a chelating agent, an antigen, a peptide, a metal, a λ or β emitting radioactive isotope, and an antibody molecule.

4- The peptide according to claim 3 wherein the therapeutic agent is selected from the group consisting of: chemotherapeutic agent, antibiotic agent, anti-inflammatory agent, anthistamine agent, antiparasitic agent, antiviral agent, and antitumor antibody.

5- The peptide according to claim 4 wherein the chemotherapeutic agent is selected from the group consisting of: cisplatin, idarubicin, epirubicin, cytarabine, farmarubicin, and etoposside.

6- The peptide according to claim 4 wherein the antibiotic agent is selected from the group consisting of: b-lactam, and quinolone antibiotics.

7- (canceled)

8- (canceled)

9- (canceled)

10- (canceled)

11- (canceled)

12- A method for inducing an immunological response in a patient comprising administering a peptide according to claim 1 thereto.

13- A method for producing antibodies in a patient comprising administering a peptide according to claim 1 thereto.

14- A molecule selected from the group consisting of:
a nucleotide sequence coding for the peptide consisting of the sequence AVCGTG (SEQ ID No. 1) or ortolog nucleotide sequence thereof;
a vector comprising said nucleotide sequence; and
a host cell genetically engineered expressing said peptide.

15- A composition comprising a molecule and acceptable excipients wherein the molecule is selected from the group consisting of:
a) peptide consisting of the sequence AVCGTG (SEQ ID No. 1);
b) a nucleotide sequence coding for the peptide consisting of the sequence AVCGTG (SEQ ID No. 1) or ortholog nucleotide sequence thereof;
c) a vector comprising said nucleotide sequence; and
d) a host cell genetically engineered expressing said peptide.

16- The composition according to claim 15 wherein the peptide is conjugated to a molecule.

17- (canceled)

18- (canceled)

19- (canceled)

20- (canceled)

21- (canceled)

22- (canceled)

23- Kit for the diagnosis of a tumor comprising the peptide according to claim 1.

24- A method for the treatment of a tumor in a patient, comprising administering a composition according to claim 15 to said patient.

25- The method of claim 24, wherein the tumor is a leukemia.

26- A method for the treatment of a tumor in a patient, comprising administering the peptide conjugated to a molecule of claim 2 to said patient.

27- The method of claim 26, wherein the tumor is a leukemia.

28- A method transporting a molecule to a target site in a patient, comprising administering the peptide conjugated to a molecule of claim 2 to said patient.

29- The method of claim 26, wherein the target site is selected from the group consisting of: tumor cell, leukocyte, gamete, normal cell, stem cell and bacteria.

30- A method for inducing an immunological response in a patient comprising administering the peptide conjugated to a molecule of claim 2 to said patient thereto.

31- A method for producing antibodies in a patient comprising administering the peptide conjugated to a molecule of claim 2 to said patient thereto.
